(19) United States
(12) Patent Application Publication
(10) Pub. No.: US 2022/0221466 A1
(43) Pub. Date: Jul. 14, 2022

(54) USE OF CAP-SCORE™ IN IDENTIFICATION OF A REPRODUCTIVE APPROACH IN MEN SUFFERING FROM VARICOCELE

(71) Applicant: ANDROVIA LIFESCIENCES, LLC, Mountainside, NJ (US)

(72) Inventors: Charles G. OSTERMEIER, Gillette, NJ (US); Alexander TRAVIS, Ithaca, NY (US)

(73) Assignee: ANDROVIA LIFESCIENCES, LLC, Mountainside, NJ (US)

(21) Appl. No.: 17/607,773

(22) PCT Filed: Apr. 30, 2020
(86) PCT No.: PCT/US2020/030783

§ 371 Date: Oct. 29, 2021

Related U.S. Application Data
(60) Provisional application No. 62/840,846, filed on Apr. 30, 2019.

(51) Int. Cl. G01N 33/68; A61B 5/00

(52) U.S. Cl. CPC G01N 33/689; G01N 2800/367; A61B 5/4387

(57) ABSTRACT

The present disclosure provides methods for identifying a patient for varicocele correction. The method includes using the CAP-Score™ Assay to assess whether fertility is affected by the varicocele. The present disclosure also provides methods for identifying a reproductive approach inpatients suffering from varicocele. The method includes using the CAP-Score™ Assay to identify and administer the reproductive approach.

### CROSS REFERENCE TO RELATED APPLICATIONS

This application claims priority to U.S. Provisional Application No. 62/840,846, filed Apr. 30, 2019, which is herein incorporated by reference in its entirety.

### BACKGROUND OF THE INVENTION

Varicoceles are a known cause of male infertility. They occur when certain veins in the scrotum become enlarged. Varicoceles can affect fertility by impairing sperm either in morphology, chromatin integrity, motility, function, or some combination thereof, although it is unclear exactly how varicoceles render sperm impaired. In any event, it has been demonstrated that fertility can be improved by surgical correction of varicoceles.

The CAP-Score™ Assay, developed by Androvia LifeSciences, is a test that measures male fertility. The CAP-Score™ Assay is a measure of sperm capacitation, and is expressed as the percentage of sperm that capacitate. Capacitation is the process that sperm must undergo in order to be capable of fertilizing an egg. Sperm capacitation can be assessed visually using G_{M1} localization patterns. In particular, the apical acrosome (AA) G_{M1} localization patterns and acrosomal plasma membrane (APM) G_{M1} localization patterns have been associated with capacitation in human sperm. In addition to the AA and APM G_{M1} localization patterns, there are other labeled localization patterns including the Lined-Cell, intermediate (INTER), post acrosomal plasma membrane (PAPM), apical acrosome/post acrosome (AA/PA), equatorial segment (ES), and diffuse (DIFF) G_{M1} localization patterns; however these patterns are not associated with capacitation. (Travis et al., “Impacts of common semen handling methods on sperm function,” The Journal of Urology, 195 (4), e909 (2016)).

The Cap-Score™ Assay is defined as ([number of AA G_{M1} localization patterns+number of APM G_{M1} localization patterns]/total number of G_{M1} labeled localization patterns), or in simpler terms, the number of capacitated sperm divided by the total number of G_{M1} localization patterns, multiplied by 100% to obtain a percentage of capacitated sperm. A CAP-Score™ of 35 or higher indicates a normal fertility status. The CAP-Score™ Assay is described, for example, in U.S. Patent Publication No. 2017/0248584, U.S. Patent Publication No. 2017/0184605 and U.S. Patent Publication No. 2017/0234857, each of which are herein incorporated by reference in their entireties.

### SUMMARY OF THE INVENTION

In one embodiment, a method for identifying a reproductive approach in a subject suffering from a varicocele is disclosed. In an embodiment, the method comprises receiving information that the subject is diagnosed with varicocele; obtaining a sperm sample, fixing said sperm sample with a fixative, labeling said sperm sample with a label that identifies G_{M1} localization patterns, measuring a total number of G_{M1} localization patterns expressed in the sperm sample (“total G_{M1}”), measuring apical acrosome (AA) and acrosomal plasma membrane (APM) G_{M1} localization patterns expressed in the sperm sample, and obtaining a CAP-Score™ as a function of total G_{M1}, AA, and APM, in particular, using AA+APM/total G_{M1} localization patterns; and providing instructions to use the recommended reproductive approach. In an embodiment of the invention, the reproductive approach is selected from varicocele repair surgery, natural conception, intrauterine insemination (IUI), in vitro fertilization (IVF), intracervical insemination (ICI), intracytoplasmic sperm injection (ICSI), gamete intra-fallopian transfer (GIFT) and subzonal insemination (SUZI). In an embodiment of the invention the varicocele is diagnosed by physical examination. In an embodiment of the invention, the reproductive approach is selected from varicocele repair surgery, IUI, ICI, IVF, ICSI, GIFT, and SUZI when the CAP-Score™ is within sub-fertile range, or less than one standard deviation below or lower than a reference mean value. In an embodiment of the invention, the reproductive approach is selected from natural conception, ICI, and IUI when the CAP-Score™ within normal fertility range, generally greater than about one standard deviation below a reference mean value. In an embodiment of the invention, the reproductive approach is varicocele repair surgery. In an embodiment of the invention, instructions are provided to the subject. In another embodiment, instructions are provided to a healthcare professional.

In an embodiment of the present invention, the method further comprises providing a post-surgery CAP-Score™ on a sperm sample obtained after varicocele repair surgery to determine whether the varicocele repair surgery is successful. In an embodiment, the method further comprises comparing the original CAP-Score™ generated before surgery to the post-surgery CAP-Score™. In an embodiment, an increase in the post-surgery CAP-Score™ as compared to the initial CAP-Score™ indicates successful variocele repair surgery. In an embodiment, an increase in the post-surgery CAP-Score™ of at least seven percent indicates successful varicocele repair surgery.

In an embodiment of the invention, the G_{M1} localization patterns are selected from apical acrosome (AA), acrosomal plasma membrane (APM), lined-cell, intermediate (INTER), post acrosomal plasma membrane (PAPM), equatorial segment (ES) and diffuse (DIFF). In an embodiment of the invention, the sperm sample is fixed for a period of at least 30 minutes. In an embodiment of the invention, the label is a fluorescence label. In another embodiment, the label is cholera toxin b subunit. In another embodiment of the invention, the method further comprises analyzing one or more sperm characteristics selected from motility, morphology, volume, concentration, pH, viscosity, and combinations thereof.

### BRIEF DESCRIPTION OF THE DRAWINGS

The foregoing summary, as well as the following detailed description of the invention, will be better understood when read in conjunction with the appended drawings.

FIG. 1 illustrates relative fertility and corresponding probability of generating a pregnancy within 3 attempts (star). The continuum was generated and tested using data from multiple patients from multiple clinical sites.

FIG. 2, comprising FIGS. 2A-2L, illustrate data from Example 2 for pre- and post-varicocelectomy ejaculates. FIGS. 2A-2D represent data from non-responders, FIGS. 2E-2H represent data from responders, and FIGS. 2I-2L represent box whisker plots illustrating mean (+) in both pre- and post varicocelectomy samples. FIGS. 2A, 2D, and 2I represent CAP-Score™ measurement, FIGS. 2B, 2E, and 2J represent sperm concentration, FIGS. 2C, 2F, and 2K represent sperm motility, and FIGS. 2D, 2G, and 2L represent normal sperm morphology.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

With reference to the accompanying drawings, various embodiments of the present invention are described more fully below. Some but not all embodiments of the present invention are shown. Indeed, various embodiments of the invention may be embodied in many different forms and should not be construed as limited to the embodiments expressly described. It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

### Definitions

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

G_{M1} refers to monosialotetrahexosylganglioside and is a member of the ganglio series of gangliosides. The term “G_{M1} localization pattern” is used interchangeably herein with G_{M1} pattern, G_{M1} localization, pattern, and/or localization pattern.

For human sperm, eight different G_{M1} localization patterns have been reported when the sperm was under in vitro capacitating conditions. To visualize the localization patterns, the capacitated sperm were treated with labeling molecule for G_{M1}, such as cholera toxin b, which has a florescence detectable label on it. The labeled sperm cells are then visualized using a fluorescence microscope as known to those of skill in the art.

INTER is characterized by a sperm cell where the vast majority of the fluorescence signal is in a band around the equatorial segment, with some signal in the plasma membrane overlying the acrosome. There is usually a gradient of the fluorescence signal, with the most at the equatorial segment and then progressively less toward the tip. There is often an increase in fluorescence signal intensity on the edges of the sperm head in the band across the equatorial segment.

Apical Acrosome “AA” is characterized by a sperm cell where the fluorescence signal is concentrated toward the apical tip, increased in brightness and reduced in area with signal.

Acrosomal Plasma Membrane “APM” is characterized by a sperm cell exhibiting a distributed fluorescence signal in the plasma membrane overlying the acrosome. APM signal is seen either from the bright equatorial INTER band moving apically toward the tip, or it can start further up toward the tip and be found in a smaller region, as it is a continuum with the AA.

Post-Acrosomal Plasma Membrane “PAPM” is characterized by a sperm cell where the fluorescence signal is exclusively in the post-acrosomal plasma membrane.

Apical Acrosome Post-Acrosome “AA/PA” is characterized by a sperm cell where the fluorescence signal is located both in the plasma membrane overlying the acrosome and post-acrosomal plasma membrane. The equatorial segment does not exhibit a fluorescence signal.

Equatorial Segment “ES” is characterized by a sperm cell having a bright fluorescence signal located solely in the equatorial segment. It may be accompanied by thickening of the sperm head across the equatorial region.

Diffuse “DIFF” is characterized by a sperm cell having a diffuse fluorescence signal located over the whole sperm head.

Lined-Cell is characterized by a sperm cell having a diffuse fluorescence signal ontop of the post-acrosomal region and at the plasma membrane overlying the acrosome as well as the bottom of the equatorial segment (i.e., the post acrosome/equatorial band). A fluorescence signal is missing around the equatorial segment.

“CAP-Score™” is defined as the ratio of [the number of apical acrosome (AA) G_{M1} localization patterns+the number of acrosomal plasma membrane (APM) G_{M1} localization patterns] divided by [the total number of G_{M1} labeled localization patterns.] (Travis et al., “Impacts of common semen handling methods on sperm function,” The Journal of Urology, 195 (4), e909 (2016)). To arrive at the total number of different G_{M1} localization patterns, the number of localization patterns are counted for at least 100 sperm cells. CAP-Score is also discussed and referred to in U.S. Patent Application Nos. 2017/0184605 and U.S. Patent Application Nos. 2017/0234857, each of which are herein incorporated by reference in their entireties.

For the purposes of this application “insemination” is understood to have a meaning dependent upon the reproductive approach. For example, for “intercourse,” insemination is understood to mean introduction of sperm into a female's reproductive tract. For example, for “intracervical insemination (ICI),” insemination is understood to mean introduction of sperm into a female's cervix. For “intrauterine insemination (IUI),” insemination is understood to mean when sperm is introduced into a female's uterus. For “in vitro fertilization (IVF),” insemination is understood to mean when sperm are introduced into a droplet of medium containing egg cells (oocytes) to allow co-incubation of sperm and egg cell(s). For “intracytoplasmic sperm injection (ICSI),” insemination is understood to mean injection of sperm or pre-capacitated sperm into an egg cell. For “gamete intra-fallopian transfer (GIFT),” insemination is understood to mean injection of sperm or pre-capacitated sperm and egg cell(s) into the female's Fallopian tubes. For “subzonal insemination (SUZI),” insemination is understood to mean injection of a single sperm cell or a single pre-capacitated sperm cell just beneath the zona pellucida. For purposes of this application, the term “cryopreservation” refers to the entire process of freezing, storing, and thawing the cells for use.

As used herein below, the male is a mammal. In an embodiment, the male is a human. In another embodiment, the male is a non-human mammal. In one such embodiment, the male is a companion animal. In another embodiment, the male is an agricultural animal. In one such embodiment, the male is a canine, feline, equine, bovine, sheep, goat, pig, camellid, or buffalo.

The term “capacitated” sperm refers to sperm which have been incubated under conditions which promote the process of capacitation. Capacitated sperm have acquired the ability to undergo acrosome exocytosis and have acquired a hyperactivated pattern of motility. Capacitated sperm are able to fertilize an egg.

CAP-Score™ Analysis

The present disclosure is based on the observations that certain G_{M1} localization patterns can provide information regarding male fertility status. Determination of G_{M1} localization patterns is described in U.S. Pat. Nos. 7,160,676, 7,670,763, and 8,367,313, the disclosures of which are incorporated herein by reference. The present disclosure provides methods for determination of a reproductive approach in a male suffering from varicocele. In certain embodiments, the method is based on a change in the percentage of certain G_{M1} localization patterns upon exposure to in vitro capacitating stimuli.

In an embodiment of the invention, CAP-Score™ is calculated by assessing [the number of apical acrosome (AA) G_{M1} localization patterns+the number of acrosomal plasma membrane (APM) G_{M1} localization patterns] divided by [the total number of G_{M1} labeled localization patterns]. In an embodiment of the invention, a semen sample is obtained, the semen sample is exposed to capacitation conditions, fixed, labeled, and visually inspected to identify G_{M1} localization patterns.

In one embodiment, capacitation conditions include the presence in the medium of one or more of bicarbonate ions, calcium ions, and a sterol acceptor, e.g., serum albumin or a cyclodextrin. In one embodiment, in vitro capacitation conditions include the presence of bicarbonate and calcium ions in the medium, and the presence of a sterol acceptor. In one embodiment, a sterol acceptor is a mediator of sterol efflux.

In one embodiment, capacitation may be induced in vitro by exposure to external stimuli such as bicarbonate and calcium ions, and mediators of sterol efflux, e.g., 2-hydroxy-propyl-β-cyclodextrin, methyl-β-cyclodextrin, serum albumin, high density lipoprotein, phospholipids vesicles, liposomes, etc. In certain embodiments, an identifiable change in the G_{M1} localization pattern is observed when sperm are exposed to one or more of these stimuli in vitro.

In one embodiment, after collection, semen samples are typically processed in some way, including one or more of the following: liquefaction, washing, and/or enrichment. In some embodiments, liquefaction involves allowing the sample to liquefy at room temperature or at 37° C. (or any temperature there between) for various time periods (typically 15-20 minutes, but ranging from 10-60 minutes). Liquefaction is a process through which the seminal plasma converts from a gel into a more fluid/liquid consistency. Seminal plasma will typically liquefy without any manipulation, but with especially viscous samples, or if there is a desire to hasten the process or make a consistent liquefaction protocol by which all samples are handled, individuals might manipulate the sample to achieve liquefaction. In certain embodiments the semen sample is manipulated to decrease semen viscosity by using a wide orifice pipette made of non-metallic material. In some embodiments the wide orifice pipette has a gauge size of at least 18 gauge, 16 gauge or 14 gauge. In some embodiments, the wide orifice pipette has an orifice size of at least 1 mm, 1.2 mm or 1.4 mm. In certain embodiments, one can also achieve liquefaction by adding various reagents which do not damage sperm membrane. Reagents which should be avoided are those that damage sperm membrane. The sperm can be washed by centrifugation and resuspension and subjected to semen analysis, and/or be subjected to one or more selection processes including: layering on top of, and centrifugation through a density gradient; layering on top of, and centrifugation through a density gradient followed by collection of the sperm-enriched fraction followed by resuspension and washing; layering on top of, and centrifugation through a density gradient followed by collection of the sperm-enriched fraction and overlaying on top of that a less dense medium into which motile sperm will swim up; or overlaying a less dense medium on top of the sample and allowing motile sperm to swim up into it.

In one embodiment, after collection, the semen sample is added to a semen extender medium as described, for example, in International PCT Application No. PCT/US2019/059477, which is herein incorporated by reference in its entirety. In an embodiment, the semen extender medium is washed from the semen sample and processed as described herein.

In one embodiment, after initial processing, the sperm are counted, and a given number of sperm are placed into containers (such as tubes) containing capacitating medium to achieve desired final concentrations. In one embodiment, the final typical concentration of sperm is 1,000,000/ml (final concentration ranges might vary from 250,000/ml to 250,000,000/ml). In one embodiment, the sperm are placed into containers containing non-capacitating medium.

In one embodiment, the capacitating medium is a physiological buffered solution, for example, human tubal fluid (HTF); modified human tubal fluid (mHTF); Whitten's medium; modified Whitten's medium; KSOM; phosphate-buffered saline; HEPES-buffered saline; Tris-buffered saline; Ham's F-10; Tyrode's medium; modified Tyrode's medium; TES-Tris (TEST)-yolk buffer; or Biggers, Whitten and Whittingham (BWW) medium. In one embodiment, the base medium includes one or more defined or complex sources of protein or other factors, including fetal cord serum ultrafiltrate, plasmanate, egg yolk, skim milk, albumin, lipoproteins, or fatty acid binding proteins, either to promote viability or at concentrations sufficient to help induce capacitation. In an embodiment of the invention, typical stimuli for capacitation include one or more of the following: bicarbonate (typically at 20-25 mM, with ranges from 5-50 mM), calcium (typically at 1-2 mM, with ranges from 0.1-10 mM), and/or cyclodextrin (typically at 1-3 mM, with ranges from 0.1-20 mM). Cyclodextrins may comprise 2-hydroxy-propyl-β-cyclodextrin and/or methyl-β-cyclodextrin.

In an embodiment of the invention, incubation in the capacitation medium takes place at a temperature of between 30° C. and 38° C. In an embodiment, the temperature is 37° C. In an embodiment, incubation in the capacitation medium takes place for from about 15 minutes to about 36 hours or more. In an embodiment of the invention, incubation times range from about 1 hour to about 4 hours. In another embodiment, incubation time is about 3 hours. In another embodiment, incubation time is about 24 hours. In another embodiment, incubation time is about 36 hours.

In one embodiment, for generating G_{M1} patterns, the sperm are washed with a standard base medium (e.g., phosphate-buffered saline, Modified Whitten's medium, or other similar media). Since G_{M1} has extracellular sugar residues which can serve as an epitope, it can be visualized without having to fix and permeabilize the cells.

In another embodiment, the sperm are fixed. Fixation of the sperm cells results in better preservation of the specimen, easier visualization (compared to discerning patterns in swimming sperm) and allows longer visualization time. Various fixatives known for histological study of spermatozoa are within the purview of those skilled in the art. Suitable fixatives include paraformaldehyde, glutaraldehyde, Bouin's fixative, and fixatives comprising sodium cacodylate, calcium chloride, picric acid, tannic acid and like. In one embodiment, paraformaldehyde, glutaraldehyde or combinations thereof are used.

In an embodiment of the invention, the fixative is paraformaldehyde. In an embodiment, paraformaldehyde is used to fix the sperm at about 0.004% (weight/volume) to about 4% (weight/volume) paraformaldehyde. In another embodiment, paraformaldehyde is used at about 0.01% to about 1% (weight/volume) to fix the sperm. In one embodiment, about 0.005% (weight/volume) paraformaldehyde to about 1% (weight/volume) paraformaldehyde is used. In one embodiment, a combination of paraformaldehyde, glutaraldehyde, and CaCl₂ is used to fix the sperm. In an embodiment, about 4% paraformaldehyde (weight/volume), about 0.1% glutaraldehyde (weight/volume) and about 5 mM CaCl₂ in phosphate buffered saline is used.

The period of time a sperm sample is fixed in a fixative may vary. In one embodiment, a sperm sample is fixed in fixative for at least about 15 minutes, about 20 minutes, about 25 minutes, about 30 minutes, about 35 minutes, about 40 minutes, about 45 minutes, about 50 minutes, about 55 minutes, or about 60 minutes. In another embodiment, the sperm sample is fixed in a fixative for about 5 hours or less. In one embodiment, a sperm sample is fixed in a fixative for greater than about 5 hours. In another embodiment, a sperm sample is fixed in a fixative for about 0.5 hours, for about 1 hours, for about 1.5 hours, for about 2 hours, for about 2.5 hours, for about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 27.5 hours, about 28 hours, about 28.5 hours, about 29 hours, about 29.5 hours, about 30, or any range determinable from the preceding times (for example, about 26 hours to about 28 hours, or about 3 hours to about 5 hours).

In one embodiment, the G_{M1} localization pattern in live or fixed sperm is obtained by using labeling/binding techniques. In one embodiment, a molecule having specific affinity for the G_{M1} ganglioside can be used. In another embodiment, the labeling molecule can be directly linked to a detectable label (such as a fluorophore) or may be detected by a second labeling molecule which has a detectable label on it. For example, the b subunit of cholera toxin is known to specifically bind to G_{M1}. Therefore, a labeled (such as fluorescent labeled) cholera toxin b subunit can be used to obtain a G_{M1} localization pattern. In one embodiment, the final concentration of the b subunit of cholera toxin linked to fluorophore ranges from about 10 μg/ml to about 15 μg/ml. In another embodiment, the final concentration of the b subunit of cholera toxin linked to fluorophore ranges from about 0.1 μg/ml to about 50 μg/ml.

In another embodiment, a labeled antibody to G_{M1} can be used. In yet another alternative, a labeled antibody to the cholera toxin b subunit can be used to visualize the pattern of G_{M1} staining. In another embodiment, a labeled secondary antibody which binds to either the primary antibody that binds directly to G_{M1} or to the primary antibody that binds to the b subunit of cholera toxin could be used. The term “G_{M1} staining” or “staining of G_{M1}” or “labeling” or related terms as used herein means the staining seen on or in cells due to the binding of labeled affinity molecules to G_{M1}. For example, when fluorescent tagged/labeled cholera toxin b subunit is used for localization of G_{M1}, the signal or staining is from the cholera toxin b subunit but is indicative of the location of G_{M1}. The terms “signal” and “staining” and “labeling” are used interchangeably. The detectable label is such that it is capable of producing a detectable signal. Such labels include a radionuclide, an enzyme, a fluorescent agent or a chromophore. Labeling (or staining) and visualization of G_{M1} distribution in sperm is carried out by standard techniques. In an embodiment of the invention, labeling molecules other than the b subunit of cholera toxin can also be used, for example, polyclonal and monoclonal antibodies. Specific antibodies to G_{M1} ganglioside can be generated by routine immunization protocols, or can be purchased commercially (e.g., Matreya, Inc., State College, Pa.). The antibodies may be raised against G_{M1} or, can be generated by using peptide mimics of relevant epitopes of the G_{M1} molecule. Identification and generation of peptide mimics is well known to those skilled in the art. In addition, the binding of the b subunit to cholera toxin might be mimicked by a small molecule. Identification of small molecules that have similar binding properties to a given reagent is well known to those skilled in the art.

Fertility Thresholds and Recommendation for Reproductive Approach

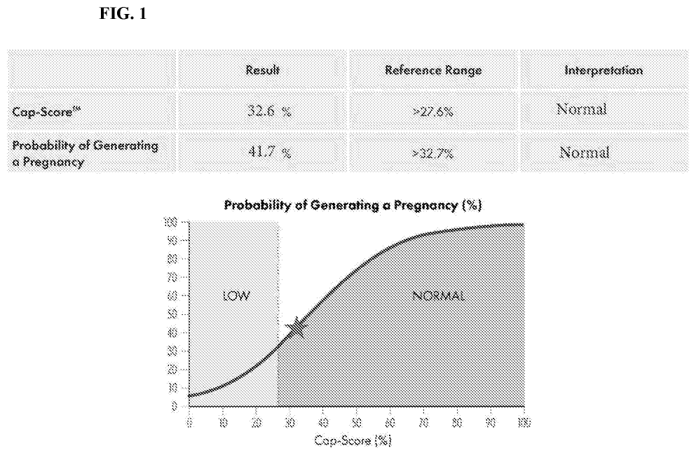

In an embodiment of the invention, a fertility threshold associated with a CAP-Score™ is determined based on distribution statistics of a known fertile reference population. A fertility threshold corresponding to greater than one standard deviation below the reference population mean CAP-Score™ indicates normal male fertility and a fertility threshold that is one standard deviation below the reference population mean or lower indicates sub-fertile fertility status. FIG. 1 illustrates a reference population of fertile males. FIG. 1 shows an exemplary reference value of 27.6%, which is one standard deviation below the mean value (35.36%, indicated by a star) of a normal, fertile male. Samples having a CAP-Score™ results above this value are considered to be in the “normal fertility” range. A CAP-Score™ that is at or lower than one standard deviation below the mean value of the reference population (i.e., at or lower than 27.6% in FIG. 1) is referenced as the “sub-fertile” range. The actual percentage values should not be construed to be a strict “cutoff” between the lowest normal fertility and sub-fertile thresholds because data are continually added to the reference population to more precisely hone the mean value, and the CAP-Score™ best measures male fertility in the form of “probability of generating a pregnancy” as shown in FIG. 1. The fertility threshold between low and normal should be characterized as stated above, that is, normal fertility status is greater than one standard deviation below the reference mean, and sub-fertile fertility status is one standard deviation below the reference mean or lower.

One of skill in the art will appreciate that the recommendation for a reproductive approach is based on many factors, including the fertility status of the male, the fertility status of the female, and other variables. In the present application, recommendation for a reproductive approach is based on the characteristics of male fertility. For example, a male with a CAP-Score™ in the “normal fertility” range would typically be recommended a reproductive approach such as natural conception, IUI, or ICI. A male with a CAP-Score™ in the sub-fertile range may be recommended a more aggressive reproductive approach such as IVF, ICSI, SUZI, or GIFT.

A varicocele itself may be the reason the CAP-Score™ falls within a sub-fertile range. If this is the case, the recommendation for reproductive approach may be to first try varicocele repair surgery, and then reassess the CAP-Score™. If the repair surgery is successful, the CAP-Score™ should increase. In one embodiment, the CAP-Score™ increases to the normal fertility threshold. In another embodiment, the CAP-Score™ does not increase to the normal fertility threshold. To the extent the CAP-Score™ increases, but not to within the normal fertility range, the recommendation for reproductive approach is similar to the recommendation for any sub-fertile male, that is, IVF, SUZI, or GIFT. If the CAP-Score™ increases to within the normal fertility range, the recommendation for reproductive approach would include natural conception, IUI, or ICI.

Varicocele and CAP-Score™

It has been identified that patients suffering from varicocele may not necessarily have significantly different semen analysis as compared to a control group (see Example 1). Thus, if a healthcare provider uses only the traditional semen analysis parameters such as volume, concentration, motility, and morphology, varicocele may not be identified as a hindrance to male fertility/reproduction and its repair may not be identified as a measure to improve fertility. With CAP-Score™ analysis, it has been shown that varicocele has a significant effect on sperm capacitation. Table 1 shows a significant difference in CAP-Score™, total sperm capacitation, and probability of generating a pregnancy between varicocele patients and control patients, indicating that repair of varicocele can improve capacitation, thus improving fertility of the male patient.

In an embodiment of the invention, the CAP-Score™ is useful in a method for assessing whether a male suffering from varicocele is a candidate for varicocele repair surgery in order to improve fertility. The method includes obtaining information that the male is suffering from varicocele, assessing the CAP-Score™ of said male, wherein if the CAP-Score™ is in the subfertile range, the male is a candidate for varicocele repair surgery in order to improve fertility.

In an embodiment of the invention, the CAP-Score™ is useful in a method for assessing whether varicocele repair surgery is successful. In an embodiment, the CAP-Score™ is assessed pre-surgery and post-surgery, wherein an increase in the post-surgery CAP-Score™ indicates a successful surgery. In an embodiment of the invention, the increase in post-surgery CAP-Score™ over pre-surgery CAP-Score™ is at least enough to account for normal variation in CAP-Score™ In an embodiment of the invention, the increase in post-surgery CAP-Score™ over pre-surgery CAP-Score™ is at least 7.7%. In an embodiment of the invention, the increase in post-surgery CAP-Score™ over pre-surgery CAP-Score™ is at least 5%, 6%, 7%, 8%, 9%, 10%, 11%, 12%, 13%, 14%, 15%, 16%, 17%, 18%, 19%, 20%, 21%, 22%, 23%, 24%, 25% or more. In an embodiment of the invention, the increase in post-surgery CAP-Score™ over pre-surgery CAP-Score™ is at least about 7.7%. In an embodiment of the invention, the increase in post-surgery CAP-Score™ over pre-surgery CAP-Score™ is at least about 6% to about 20%. In an embodiment of the invention, the increase in post-surgery CAP-Score™ over pre-surgery CAP-Score™ is at least 7% to about 15%. In an embodiment of the invention, the increase in post-surgery CAP-Score™ over pre-surgery CAP-Score™ is at least about 7%. In an embodiment of the invention, the increase in post-surgery CAP-Score™ over pre-surgery CAP-Score™ is at least about 12%. In an embodiment of the invention, an increase in CAP-Score™ between pre- and post-varicocelectomy may indicate a successful surgery, and the post-surgery CAP-Score™ may move into the normal fertility range. In such a case, a reproductive approach such as natural conception, IUI, or ICI may be recommended. In an embodiment of the invention, an increase in CAP-Score™ between pre- and post-varicocelectomy may indicate a successful surgery, but the CAP-Score™ may still be within the subfertile range. In such a case, a reproductive approach such as ICI, IVF, GIFT, SUZI, or ICSI may be recommended.

In an embodiment of the invention, a method for identifying a reproductive approach in a subject suffering from varicocele is disclosed. The method includes receiving information that the subject is suffering from varicocele, assessing the CAP-Score™ in said subject, and providing instructions regarding the recommended reproductive approach. In an embodiment of the invention, varicocele repair surgery to increase the CAP-Score™, thus increasing fertility, is recommended. In another embodiment, the method further comprises assessing a post-surgery CAP-Score™ in a patient who has undergone varicocele repair surgery to identify a recommended reproductive approach. In an embodiment of the invention, an increase in post-surgery CAP-Score™ into the normal fertility range informs a recommendation for natural conception, IUI, or ICI. In an embodiment of the invention, an increase in post-surgery CAP-Score™ into the normal fertility range informs a recommendation for ICI, IVF, GIFT, SUZI, or ICSI.

### EXAMPLES

### Example 1: Effect of Varicocele on Sperm Capacitation

Study data exhibited at the 2019 American Society for Reproductive Medicine (ASRM) Scientific Congress & Expo by Philip Xie, et al. (Oct. 15, 2019; DOI: https://doi.org/10.1016/j.fertnstert.2019.07.793) show that varicoceles affect sperm capacitation. Ejaculates from 18 men were obtained and traditional semen analysis was performed. CAP-Score™ was determined using a proprietary method developed by Androvia LifeSciences, as described in U.S. Patent Application Nos. 2017/0184605 and U.S. Patent Application Nos. 2017/0234857. Briefly, each ejaculate was incubated in capacitation medium for a period of 3 hours. The ejaculate was then fixed with a fixative for a period of at least 30 minutes, labeled with a fluorescence label for at least 10 minutes, then assessed for G_{M1} localization patterns. Patterns AA and APM indicate capacitated sperm. The percentage of AA+APM patterns was determined, and a CAP-Score™ was assigned. Men with a CAP-Score™ lower than one standard deviation below the mean of a population (about 35.3%) of fertile men were considered having low fertility (about 27.6% or lower).

Of the 18 men tested, 8 were diagnosed with a variocele of Grade 2 or higher by physical examination. Ten men were in the control group. Ejaculate samples were taken at the same time in the control and varicocele groups.

The results of the semen analysis between the control and varicocele groups indicated that semen parameters such as volume, concentration, motility, and morphology were not significantly different. However, CAP-Score™, total sperm capacitation, and probability of generating a pregnancy (PGP) were all significantly lower in the varicocele population (see Table 1, below), indicating for the first time that capacitation of sperm, and thus, the ability to fertilize an egg, is affected by varicocele.

TABLE 1
Results of Semen Analysis and CAP-Score ™

| | Control | Variocele | |
|---|---|---|---|
| Parameter | (n = 10) | (n = 8) | P value |
| Age (years) | 34.7 ± 2.8 | 36.0 ± 7.0 | NS |
| Volume (ml) | 2.9 ± 0.9 | 3.1 ± 1.1 | NS |
| Concentration (×10⁶/ml) | 69.8 ± 26.3 | 45.4 ± 30.9 | NS |
| Motility (%) | 47.7 ± 2.8 | 45.6 ± 1.0 | NS |
| Normal Morphology (%) | 3.1 ± 0.7 | 2.8 ± 1.0 | NS |
| CAP-Score ™ (%) | 31.4 ± 4.8 | 26.4 ± 3.7 | 0.03 |
| Total Capacitated Sperm (millions) | 67.1 ± 40.1 | 32.8 ± 22.4 | 0.04 |
| Probability of Generating a | 39.7 ± 9.1 | 31.0 ± 6.0 | 0.03 |
| Pregnancy (PGP, %) | | | |

### Example 2: Improvement of CAP-Score™ After Varicocele Surgery—Study 1

Study data exhibited by Eric Seaman, et al. at the 2019 Annual Conference of the Foundation for Reproductive Medicine (Nov. 21-24, 2019) show that CAP-Score™ improves after varicocele. Ejaculates were obtained from men diagnosed with varicocele both pre-varicocelectomy and at least three months post-varicocelectomy. Traditional semen analysis was performed as determined by the World Health Organization (WHO). WHO semen parameters include volume (ml), sperm concentration, total sperm count, motility (% progressive), vitality (% live), and morphology (% normal). CAP-Score™ was assessed using the method described in Example 1. Significance in results was determined using a paired sample t-test.

In the present example, seven men were tested, 5 of which (or 71%) showed improvement in CAP-Score™. This improvement corresponded to a 91% increase in their probability of generating a pregnancy (PGP) over three cycles. Men showing an improvement in CAP-Score™ were advised to pursue reproductive approaches including natural conception and/or IUI. Men showing no improvement in CAP-Score™ were advised to pursue IVF and/or ICSI.

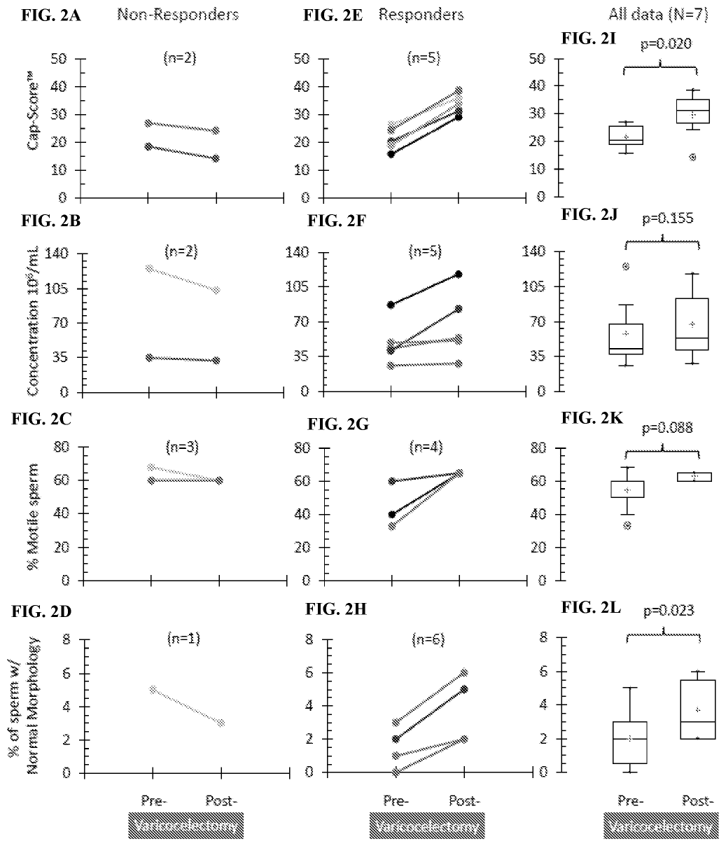

Results for responders and non-responders are shown in FIGS. 2A-2L. FIGS. 2A-2D show results for non-responders in each of CAP-Score™, concentration, motility, and normal morphology, respectively. FIGS. 2E-2H show results for responders in each of those same categories. FIGS. 2I-2L are “box whisker” plots. In these plots, the “+” indicates the mean for each of the pre- and post-varicocelectomy groups for the respective parameter, the center bar is the median, and the lower and upper limits of the box are the first and third quartiles, respectively. Points below and above the upper and lower bounds are considered outliers.

FIG. 2E shows improvement pre- and post-varicocelectomy in CAP-Score™, with an average increase of about 12.6%±1.0%, and the corresponding whisker plot in FIG. 2I shows a p value of 0.02. FIG. 2F shows improvement pre- and post-varicocelectomy in concentration, and the corresponding whisker plot in FIG. 2J shows a p value of 0.155. FIG. 2G shows improvement pre- and post-varicocelectomy in motility, and the corresponding whisker plot in FIG. 2K shows a p value of 0.088. FIG. 2H shows pre- and post-varicocelectomy in morphology, and the corresponding whisker plot in FIG. 2L shows a p value of 0.023. Three responders had a significant improvement in all three of concentration, motility and morphology, one responder had significant improvement in motility and morphology, and one responder had significant improvement in morphology only. Overall six of the seven men had some improvement in at least one semen parameter.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

We claim:

1. A method for identifying a reproductive approach in a subject suffering from a varicocele, the method comprising:
(a) receiving information that the subject is diagnosed with varicocele;
(b) obtaining a sperm sample from the subject;
(c) fixing the sperm sample with a fixative;
(d) labeling said sperm sample with a label that identifies G_{M1} localization patterns;
(e) measuring a total number of G_{M1} localization patterns expressed in the sperm sample (“total G_{M1}”), a number of apical acrosome (AA) G_{M1} localization patterns and a number of acrosomal plasma membrane (APM) G_{M1} localization patterns expressed in the sperm sample;
(f) obtaining a CAP-Score™ as a function of total G_{M1}, AA, and APM; and
(g) providing instructions to use the recommended reproductive approach.

2. The method of claim 1, wherein the varicocele is diagnosed by physical examination.

3. The method of claim 1, wherein the reproductive approach is selected from varicocele repair surgery, natural conception, intrauterine insemination (IUI), in vitro fertilization (IVF), intracervical insemination (ICI), intracytoplasmic sperm injection (ICSI), gamete intra-fallopian transfer (GIFT) and subzonal insemination (SUZI).

4. The method of claim 1, wherein the reproductive approach is selected from natural conception, IUI and ICI when the CAP-Score™ of the subject is greater than one standard deviation below a reference population mean.

5. The method of claim 1, wherein the reproductive approach is selected from IVF, ICSI, GIFT, and SUZI when the CAP-Score™ is lower than one standard deviation below a reference population mean.

6. The method of claim 1, wherein the instructions are provided to the subject.

7. The method of claim 1, wherein the instructions are provided to a healthcare professional.

8. The method of claim 1, wherein the reproductive approach is varicocele repair surgery.

9. The method of claim 8, further comprising providing a post-surgery CAP-Score™ on a sperm sample obtained after varicocele repair surgery to determine whether the varicocele repair surgery is successful.

10. The method of claim 9, further comprising comparing the CAP-Score™ to the post-surgery CAP-Score™, wherein an increase in the post-surgery CAP-Score™ of at least seven percent indicates successful varicocele repair surgery.

11. The method of claim 1, wherein the G_{M1} localization patterns are selected from apical acrosome (AA), acrosomal plasma membrane (APM), lined-cell, intermediate (INTER), post acrosomal plasma membrane (PAPM), equatorial segment (ES) and diffuse (DIFF).

12. The method of claim 1, wherein the sperm sample is fixed for a period of at least 30 minutes.

13. The method of claim 1, wherein the label is a fluorescence label.

14. The method of claim 13, wherein the label is cholera toxin b subunit.

15. The method of claim 1, further comprising analyzing one or more sperm characteristics selected from motility, morphology, volume, concentration, pH, viscosity, and combinations thereof.
